(19) United States
(12) Patent Application Publication
(10) Pub. No.: US 2013/0344081 A1
(43) Pub. Date: Dec. 26, 2013

(54) METHOD OF TREATING AMYOTROPHIC LATERAL SCLEROSIS

(71) Applicant: Ryuji Kaji, Tokushima-shi (JP)

(72) Inventor: Ryuji Kaji, Tokushima-shi (JP)

(73) Assignee: THE UNIVERSITY OF TOKUSHIMA, Tokushima (JP)

(21) Appl. No.: 14/003,494

(22) PCT Filed: Mar. 6, 2012
(86) PCT No.: PCT/JP2012/056217

§ 371 Date: Sep. 6, 2013

Related U.S. Application Data
(60) Provisional application No. 61/449,753, filed on Mar. 7, 2011.

(51) Int. Cl. A61K 39/395

(52) U.S. Cl. CPC A61K 39/3955
USPC 424/145.1

(57) ABSTRACT

Problem: An object of the present invention is to provide a drug effective for the treatment of ALS, or a method for treating ALS.

Solution: The present invention provides an ALS treatment method that improves clinical symptoms of ALS or suppresses the progression of ALS by administering an anti-TNFalfa monoclonal antibody to an ALS patient; an anti-ALS drug containing an anti-TNFalfa monoclonal antibody; an anti-TNFalfa monoclonal antibody for use as an anti-ALS drug; and use of an anti-TNFalfa monoclonal antibody for the treatment of ALS and for the manufacture of a medicament.

### TECHNICAL FIELD

The present invention relates to a method for treating amyotrophic lateral sclerosis (hereinafter referred to as ALS), and a drug used therefor.

### BACKGROUND ART

ALS is a rapidly progressive disease that shows symptoms such as muscle atrophy and muscle weakness. About 3 to 5 years after the onset of ALS, the symptoms occur in the respiratory muscles, causing death due to respiratory failure, unless a mechanical ventilation system is provided. Muscle atrophy seen in ALS patients characteristically shows abnormal excitation (muscle spasticity or fasciculation) of muscles and motor neurons. Further, ALS patients are characteristically free from the following symptoms: atrophy of the muscles controlled by sensory nerves, autonomic nerves, and the like; abnormality in eye movement; and disorder in the functions of rectum, bladder, and the like.

According to the epidemiological investigation, there is no significant difference in the incidence of ALS among ethnic groups. The annual incidence of ALS is known to be about two per 100,000 people. Further, although some patients develop ALS in their teens, the peak age of onset of ALS is 40 to 69. Among ALS patients, about 5 to 10% are patients with familial ALS, and the remaining majority of patients have sporadic ALS.

Among familial ALS patients, 20 to 30% of the patients carry a point mutation of superoxide dismutase 1 (SOD1) gene. It has been clear that SOD1 transgenic mice present a phenotype in which motor neurons are altered (NPL 1). The death of motor neurons is not recognized in SOD1 knockout mice.

Based on such findings, clinical conditions of motor neurons of ALS patients have been examined. Release of a significant amount of glutamic acid, which is considered to be a possible cause of muscle spasticity, is identified in motor neurons of patients with early ALS. Based on such a finding, an anti-ALS drug that prevents the death of motor neurons has been developed. Specifically, a drug that inhibits the action of glutamic acid on motor neurons has been developed. Specific examples include Rilutek (registered trademark, Aventis Pharma) that works as a glutamic acid release inhibitor, and the like. There is also a method, as one of the ALS treatment methods, in which a large amount of methylcobalamin (a vitamin B12 derivative) is administered to an ALS patient. However, these drugs, and a treatment method that administers these drugs, are not considered to sufficiently and effectively treat ALS.

As shown in NPL 2, the present inventors found, in a patient with familial ALS, a mutation of optineurin (OPTN) gene that inhibits NFκB function, which has an important role in nerve cell death. It has been also reported that a significant amount of OPTN is accumulated in motor neurons of patients with sporadic ALS. The present inventors also found that the accumulation of mutant OPTN in motor neurons is induced by overexpression of mutant OPTN by activated NFκB. Therefore, it was found that while wild-type OPTN can inhibit NFκB function, mutant OPTN found in ALS patients does not have the ability to inhibit NFκB function. This suggested that inhibition of activation of NFκB function would lead to an ALS treatment.

Conventionally, inhibitors of NFκB function have been used in ALS patients. However, among NFκB inhibitors, immunosuppressive drugs such as steroids have been reported to be ineffective (NPL 3). Thalidomide is one such steroid. Thalidomide was effective in SOD1 transgenic mice, but not in ALS patients (NPL 4). It has also been reported that simply knocking out the TNF locus of SOD1 transgenic mice does not show a treatment effect in ALS (NPL 5).

### CITATION LIST

### Patent Literatures

### PTL 1: Patent No. 3861118

### PTL 2: Patent No. 4404181

### Non-Patent Literatures

NPL 1: Gurney M E, Pu H, Chiu A Y, Dal Canto M C, Polchow C Y, Alexander D D, Caliendo J, Hentati A, Kwon Y W, Deng H X, et al., “Motor neuron degeneration in mice that express a human Cu, Zn superoxide dismutase mutation”, Science, 1994 Jun 17; 264 (5166): 1772-5.
NPL 2: Maruyama H, Morino H, Ito H, Izumi Y, Kato H, Watanabe Y, Kinoshita Y, Kamada M, Nodera H, Suzuki H, Komure O, Matsuura S, Kobatake K, Morimoto N, Abe K, Suzuki N, Aoki M, Kawata A, Hirai T, Kato T, Ogasawara K, Hirano A, Takumi T, Kusaka H, Hagiwara K, Kaji R, Kawakami H, “Mutations of optineurin in amyotrophic lateral sclerosis,” Nature; 465: 223-226.
NPL 3: Tan E, Lynn D J, Amato A A, Kissel J T, Rammohan K W, Sahenk Z, Warmolts J R, Jackson C E, Barohn R J, Mendell J R, “Immunosuppressive treatment of motor neuron syndromes: Attempts to distinguish a treatable disorder,” Arch Neurol, 1994 Feb; 51(2):194-200
NPL 4: Stommel E W, Cohen J A, Fadul C E, Cogbill C H, Graber D J, Kingman L, Mackenzie T, Channon Smith J Y, Harris B T, “Efficacy of thalidomide for the treatment of amyotrophic lateral sclerosis: a phase II open label clinical trial,” Amyotroph Lateral Scler., 2009 Oct-Dec; 10 (5-6): 393-404.
NPL 5: Gowing et al, “Absence of tumor necrosis factor-alpha does not affect motor neuron disease caused by superoxide dismutase 1 mutations,” J Neuroscience, 2006 Vol. 26: 11397-11402
NPL 6: de Carvalho M, Dengler R, Eisen A, England J D, Kaji R, Kimura J, Mills K, Mitsumoto H, Nodera H, Shefner J, Swash M., “Electrodiagnostic criteria for diagnosis of ALS,” Clin. Neurophysiol 2008; 119: 497-503.
NPL 7: “Adult-onset primary open-angle glaucoma caused by mutations in optineurin,” Rezaie T, Child A, Hitchings R, Brice G, Miller L, Coca-Prados M, Héon E, Krupin T, Ritch R, Kreutzer D, Crick RP, Sarfarazi M. Science. 2002 Feb 8; 295(5557): 1077-9.

### SUMMARY OF INVENTION

### Technical Problem

As described in the above-mentioned NPL 2, the present inventors knew that it would be possible to successfully treat ALS symptoms by inhibiting NFκB function in the cells. Therefore, the present inventors believed that NFκB inhibitors would be effective in the treatment of ALS; however, as shown in the above-mentioned NPLs 3 to 5, conventional inhibitors of NFκB function are known to show no effect in the treatment of ALS. In other words, a drug effective for the treatment of ALS has not yet been found. Therefore, a main object of the present invention is to provide an effective drug to treat ALS, or a treatment method of ALS.

### Solution to Problem

The present inventors administered a drug containing a monoclonal antibody against human tumor necrosis factor alpha (TNFα) to ALS patients. As a result, the present inventors obtained clinical findings that such administration slowed down the progression of muscle weakness observed in ALS patients, and also reduced muscle spasticity. The present invention was completed based on such clinical findings, and widely encompasses the following embodiments.

Item 1 An inhibitor of NFκB function comprising anti-TNF monoclonal antibody.
Item 2 An anti-ALS drug comprising an anti-TNFα monoclonal antibody.
Item 3 The anti-ALS drug according to Item 2, wherein the antibody has an inhibitory activity on NFκB function.
Item 4 A method for inhibiting NFκB function in mammals, comprising a step of administering an anti-TNFα monoclonal antibody to a mammal.
Item 5 A method for treating ALS, comprising a step of administering an anti-TNFα monoclonal antibody to an ALS patient.
Item 6 The method according to Item 5, wherein the antibody has an inhibitory activity on NFκB function.
Item 7 Use of an anti-TNFα monoclonal antibody as an inhibitor of NFκB function.
Item 8 An anti-TNFα monoclonal antibody for use in the treatment of ALS.
Item 9 The antibody according to Item 8, wherein the antibody
Item 10 Use of an anti-TNFα monoclonal antibody for the manufacture of an inhibitor of NFκB function.
Item 11 Use of an anti-TNFα monoclonal antibody for the manufacture of a medicament for the treatment of ALS.
Item 12 The use according to Item 11, wherein the antibody has an inhibitory activity on NFκB function.

### Advantageous Effects of Invention

The present invention improves ALS symptoms of ALS patients, or has an effect of suppressing the progression of ALS. ALS is a progressive disease that causes atrophy of the muscles controlled by motor nerves. As the progression advances, motor skills needed in daily life will be reduced; consequently, means for voluntary communication will be lost, causing a significant reduction in QOL. Ultimately, the patients will become unable to breathe through the lungs. This leads to death, unless a mechanical ventilation system is provided.

Therefore, the drug to treat ALS and the method to treat ALS provided by the present invention are extremely useful because the drug and the method can enhance human OQL, and provide a life that does not require a mechanical ventilation system.

Further, the inhibitor of NFκB function of the present invention exhibits an effect of improving ALS symptoms of ALS patients, as described above. At the same time, the inhibitor also has an effect of treating a disease induced by abnormality in the body, caused by the binding of TNFα to a TNFα receptor. Examples of such diseases include those described in PLTs 1 and 2, such as sepsis, autoimmune diseases (for example, rheumatoid-like arthritis, allergy, multiple sclerosis, autoimmune diabetes, autoimmune uveitis, nephrotic syndrome, and the like), infectious disease, malignant disease, transplant rejection or graft-versus-host disease, lung disease, bone disease, bowel disease, and heart disease.

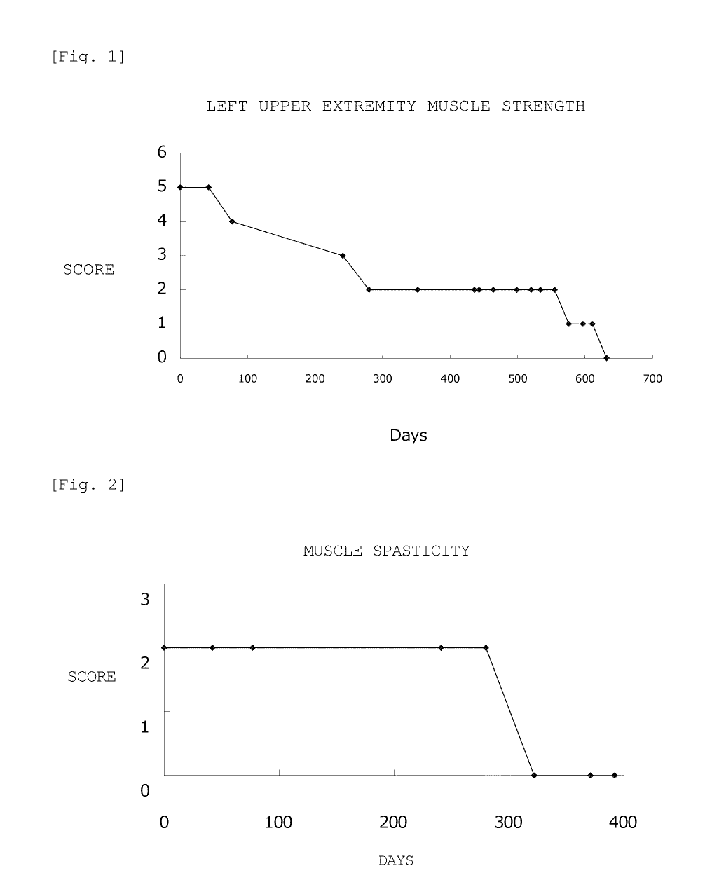

### BRIEF DESCRIPTION OF DRAWINGS

[FIG. 1] FIG. 1 shows changes in left upper extremity muscle strength of an ALS patient. The arrow in the middle of the figure shows the day when Humira (adalimumab) was administered.

The horizontal axis of the graph shows the number of days since the first visit. The vertical axis shows the score for evaluating the left upper extremity muscle strength, which is defined in the example.

[FIG. 2] FIG. 2 shows changes in muscle spasticity of an ALS patient. The arrow in the middle of the figure shows the day when Humira (adalimumab) was administered. The horizontal axis of the graph shows the number of days since the first visit. The vertical axis shows the score for evaluating the muscle spasticity, which is defined in the example.

### DESCRIPTION OF EMBODIMENTS

### Anti-TNFα Monoclonal Antibody

The anti-TNFα monoclonal antibody of the present invention is not limited as long as the monoclonal antibody recognizes TNFα as an antigen; the origin of the TNFα is also not particularly limited. Specific examples include monoclonal antibodies that recognize TNFα derived from mouse, rat, bovine, equine, porcine, human, chimpanzee, monkey, and the like as antigens. Human-derived TNFα is preferable.

The anti-TNFα monoclonal antibody of the present invention encompasses antibodies having structures of various types of immunoglobulin molecules such as IgA, IgD, IgE, IgG, IgM, and IgY. Further, the above-described IgG includes all subtypes of IgG. Further, the above immunoglobulin molecules are not limited to immunoglobulin molecules including dimers consisting of heavy and light chains. Any immunoglobulin molecule having a variable region that specifically binds to TNFα may be used. Examples thereof include immunoglobulin fragments such as Fab fragment, F(ab′)₂ fragment, Fd fragment, and Fv fragment; single-chain antibodies such as scFv and scDb; and multivalent antibodies such as diabodies, triabodies, and tetrabodies.

The origins of these antibodies are also not particularly limited. Specific examples include antibodies derived from mouse, rat, bovine, equine, porcine, human, chimpanzee, monkey, and the like. Human-derived antibodies are preferable; however, chimeric antibodies produced by combining human-derived antibodies with antibodies from different animal species (for example, mice) may also be used.

The anti-TNFα monoclonal antibody of the present invention is not particularly limited as long as it has a variable region that binds to TNFα. The amino acid sequence of a complementarity determining region (CDR) contained in such a variable region is not particularly limited. For example, reference may be made to the amino acid sequences described in PTL 1 or 2.

Specific amino acid sequences are the amino acid sequences of the CDRs contained in the variable regions of SEQ ID NOs: 3 to 8 and 11 to 35. SEQ ID NOs: 1 and 9 show the amino acid sequences of light chain variable regions comprising CDRs. SEQ ID NOs: 2 and 10 show amino acid sequences of heavy chain variable regions comprising CDRs.

CDRs having the amino acid sequences shown in any of SEQ ID NOs: 3 to 8 and 11 to 35 may be contained singly or in combination of two or more in the variable region of the monoclonal antibody of the present invention. In any case, the variable region contains at least CDR3.

Of these amino acid sequences that constitute the variable region, one of the amino acid residues at positions 1, 4, 5, 7, and 8 in the amino acid sequence of SEQ ID NO: 3 of the heavy variable region may be substituted with alanine. Further, 1 to 5 amino acid residues among amino acid residues at positions 1, 3, 4, 6, 7, 8, and 9 may be conservatively substituted.

Further, one of the amino acid residues at positions 2, 3, 4, 5, 6, 8, 9, 10, and 11 of SEQ ID NO: 4 may be substituted with alanine. Further, 1 to 5 amino acid residues among amino acid residues at positions 2, 3, 4, 5, 6, 8, 9, 10, 11 and 12 may be conservatively substituted.

The term “conservative substitution” means a substitution of an amino acid residue with another amino acid residue with a similar side chain. For example, a substitution between amino acid residues with basic side chains (lysine, arginine, and histidine) corresponds to the “conservative substitution” referred to in the present invention.

Additionally, the following substitutions also correspond to the “conservative substitution” referred to in the present invention: substitutions between amino acid residues with acid side chains such as aspartic acid and glutamic acid; substitutions between amino acid residues with non-charged polar side chains such as glycine, asparagine, glutamine, serine, threonine, tyrosine, and cysteine; substitutions between amino acid residues with nonpolar side chains such as alanine, valine, leucine, isoleucine, proline, phenylalanine, methionine, and tryptophan; substitutions between amino acid residues with β-branched side chains such as threonine, valine, and isoleucine; and substitutions between amino acid residues with aromatic side chain such as tyrosine, phenylalanine, tryptophan, and histidine.

However, these amino acid substitutions are limited within a range that does not significantly impair the specificity to anti-TNFα.

Further, as the rate constants of the antigen-antibody reaction between the anti-TNFα monoclonal antibody of the present invention and TNFα, the anti-TNFα monoclonal usually shows a K_{d} of 1×10⁻⁸M or lower, and a K of 1×10⁻³M⁻¹ or lower.

Among the above-described anti-TNFα monoclonal antibodies, the anti-TNFα monoclonal antibodies described in PTL 1 or 2 are preferable. A more preferable antibody is adalimumab, contained as an active ingredient in Humira (registered trademark, Abbott Laboratories).

Further, other preferable examples of anti-TNFα monoclonal antibodies include infliximab, contained as an active ingredient in Remicade (registered trademark, Centocor Ortho Biotech, Incorporated).

### ALS Patients of the Present Invention

ALS patients of the present invention refer to patients whose motor neurons are altered and who exhibit progressive muscle atrophy. In particular, in the present invention, preferable ALS patients are those who are at an early stage of ALS, have mild muscle atrophy, and exhibit symptoms such as muscle spasticity and fasciculation. Whether a patient has ALS can be determined using the AWAJI criteria (NPL 6) that allows diagnosis at an early stage. A patient who meets the AWAJI criteria is considered to be a preferable ALS patient in the present invention.

### Pathogenesis of ALS

The target ALS of the present invention is a disease resulting from cell death caused by the activation of NFκB, as described in NPL 2. Usually, NFκB is inhibited by OPTN. NFκB induces not only cell death, but also expression of OPTN. In other words, in general, even if NFκB is activated, OPTN is expressed, thus inhibiting NFκB. This negative feedback action strictly regulates NFκB function. Cell death caused by NFκB is regulated by such an action.

However, in the target ALS of the present invention, a mutation occurs in OPTN, and NFκB inhibitory activity by normal wild-type OPTN is thus impaired, resulting in the induction of cell death. Further, NFκB also causes induction of expression of mutant OPTN. Accordingly, mutant OPTN that cannot inhibit NFκB will be overexpressed, resulting in the induction of cell death.

OPTN contains an amino acid sequence encoded by a gene shown in NPL 7. In the case of human, OPTN is a protein encoded by a gene located on chromosome 10. Further, a gene encoding OPTN is considered to be a causative gene of open-angle glaucoma.

### NFκB Function Inhibitor

As described above, because the anti-TNFα monoclonal antibody of the present invention has an activity to inhibit NFκB function, it can be used as an inhibitor of NFκB function. Specifically, the anti-TNFα monoclonal antibody of the present invention is used to produce an inhibitor of NFκB function.

The inhibitor of NFκB function of the present invention contains the above-described anti-TNFα monoclonal antibody as an active ingredient. Insofar as the anti-TNFα monoclonal antibody is contained as an active ingredient, the inhibitor may be the antibody itself, or may contain other components. When other components are contained, the content of the anti-TNFα monoclonal antibody based on 100% by weight of the inhibitor of NFκB function is usually about 0.1 to 99% by weight.

The inhibitor of NFκB function of the present invention inhibits NFκB function as described above, thereby improving clinical symptoms of ALS or effectively suppressing the progress of the symptoms, and is thus usefully used as an anti-ALS drug. Accordingly, pharmaceutically acceptable carriers, additives, and the like are preferable components to be contained in the inhibitor of NFκB function of the present invention, together with the above-described anti-TNFα monoclonal antibody.

Examples of clinical symptoms of ALS include muscle spasticity, fasciculation, muscle atrophy, and the like that are specific to ALS patients. The inhibitor of NFκB function can be administered orally or parenterally (including intravenous (IV), intraarterial, intramuscular (IM), intracardiac, subcutaneous (SC), intraosseous, intradermal (ID), intrathecal, intraperitoneal, and intravesical routes of administration) to mammals.

Examples of mammals include human, mouse, rat, bovine, equine, porcine, human, chimpanzee, monkey, and the like, with human being preferable. Other preferable examples are rodents or small animals (such as mice, rats, and rabbits) used as experimental animals.

### Anti-ALS Drug

The anti-ALS drug of the present invention contains an anti-TNFα monoclonal antibody. In other words, an anti-TNFα monoclonal antibody can be used for the manufacture of a medicament to treat ALS. Because the inhibitor of NFκB function of the present invention can be usefully used as an anti-ALS drug, the anti-TNFα monoclonal antibody to be contained in the anti-ALS drug may be used in the same manner as described above in terms of the content and the like.

Further, the anti-ALS drug of the present invention contains an anti-monoclonal antibody, and insofar as the anti-monoclonal antibody is contained, the anti-ALS drug may be the antibody itself, or may contain other components.

The anti-ALS drug has an effect of improving the above-described clinical symptoms of ALS, or suppressing the progression of ALS. Herein, examples of clinical symptoms of ALS include muscle spasticity, fasciculation, muscle atrophy, and the like that are observed among ALS patients.

Among the above-described anti-ALS drugs, anti-ALS drugs containing adalimumab as an active ingredient are preferable, with Humira being more preferable. Other preferable embodiments include anti-ALS drugs containing infliximab as an active ingredient, with Remicade being further preferable.

The anti-ALS drug of the present invention can be preferably used in the above-described ALS patients. The drug is usually administered to the patients in an amount of 0.1 to 10 mg/kg/day, preferably in an amount of about 0.5 to 4 mg/kg/day. The dosage may be divided into several doses per day. The dosing interval is not particularly limited. The drug is usually administered once every two weeks to once every two months.

The administration method of the anti-ALS drug of the present invention is not particularly limited. Examples include intravenous (IV), intraarterial, intramuscular (IM), intracardiac, subcutaneous (SC), intraosseous, intradermal (ID), intrathecal, intraperitoneal, and intravesical routes of administration. Of these, the subcutaneous route of administration is preferable.

### ALS Treatment Method

The ALS treatment method of the present invention comprises a step of administering the anti-TNFα monoclonal antibody to an ALS patient. The ALS treatment method means to improve the above-described clinical symptoms of ALS, or suppress the progression of ALS. The ALS treatment method also has an effect of preventing the development of ALS (expression of the symptoms), and includes a treatment to maintain the status quo of a human who does not meet the criteria for ALS diagnosis, but who seems to present ALT symptoms, so that they can be prevented from reaching the level of being diagnosed as having developed ALS.

The dosage and method of administration of the anti-TNFα monoclonal antibody are as described above for the anti-ALS drug.

Further, it can also be said that such an anti-TNFα monoclonal antibody is used for the treatment of ALS.

### Method for Inhibiting NFκB Function

As described above, because the anti-TNFα monoclonal antibody has an activity to inhibit NFκB function, it can be administered to, in particular, mammals, and thereby be used to inhibit NFκB function in the mammals.

Cell death, particularly cell death of neurons, can be prevented by inhibiting specific NFκB function.

The dosage and method of administration of anti-TNFα monoclonal antibody may be the same as those described in detail for the ALS treatment method.

Hereinafter, the present invention is described in detail based on the descriptions in an example. The present invention is not limited to the example.

### EXAMPLE 1

Humira (registered trademark, Abbott Laboratories) comprising a humanized anti-human TNFα monoclonal antibody as an active ingredient was administered to an ALS patient. Clinical observations on the left upper extremity muscle strength and muscle spasticity were made.

The left upper extremity muscle strength was measured using the Medical Research Council (MRC) scale, and the measurement values were evaluated using a score shown on the vertical axis in FIG. 1.

Muscle spasticity was measured using the method suggested in NPL 6 (3: prominent, 2: large amount, 1: small amount, 0: absent). The measurement values were evaluated using a score shown on the vertical axis in FIG. 2.

A subject in this example was a sporadic ALS patient (a Japanese male in his 60s, exhibiting mild symptoms).

Clinical observations of the subject were obtained by diagnosis and medical examination on Jan. 9, 2010 (first visit); Feb. 20, 2010; Mar. 27, 2010; Sep. 7, 2010; Oct. 16, 2010; Dec. 27, 2011; Jan. 8, 2010; Jan. 15, 2011; Feb. 5, 2011, Mar. 12, 2011; Apr. 2, 2011; Apr. 16, 2011; May 7, 2011; May 28, 2011; Jun. 18, 2011; Jul. 2, 2011; Jul. 23, 2011; and Sep. 24, 2011.

Further, Humira was administered on Dec. 27, 2010; Jan. 15, 2011; Feb. 5, 2011; Mar. 12, 2011; Apr. 2, 2011; Apr. 16, 2011; May 7, 2011; May 28, 2011; Jun. 18, 2011; Jul. 2, 2011; and Sep. 24, 2011. Humira was administered via hypodermic injection. A total of 80 mg/day of Humira was administered to the subject with a body weight of 72 kg.

FIGS. 1 and 2 show clinical observations of the subject. FIG. 1 shows the left upper extremity muscle strength using the above-described score. The score showed the tendency of decrease until the administration of Humira, and the left upper extremity muscle strength reduced along with the decrease in the score. After Humira was administered, the decrease in the score tended to be alleviated. Accordingly, it became clear that Humira has an effect of suppressing a symptom, i.e., left upper extremity weakness, in the ALS patient.

The death of the subject was confirmed by the attending neurologist (R.K.) on Sep. 30, 2011. When Humira was not administered to the subject, the period of death would be assumed to be about 260 days before the actual death from a viewpoint of the attending neurologist (R.K.).

FIG. 2 shows the muscle spasticity using the above-described score. Until the administration of Humira, the score was maintained and the score showed the tendency of ongoing occurrence of muscle spasticity. However, after administration of Humira, the score reached zero, making it clear that there was no occurrence of muscle spasticity. Accordingly, it became clear that Humira improves the symptom, i.e., muscle spasticity, in the ALS patient.

1-3. (canceled)

4. A method for inhibiting NFκB function in mammals, comprising a step of administering an anti-TNFα monoclonal antibody to a mammal.

5. A method for treating ALS, comprising a step of administering an anti-TNFα monoclonal antibody to an ALS patient.

6. The method according to claim 5, wherein the antibody has an inhibitory activity on NFκB function.

7-14. (canceled)


SEQUENCE LISTING (35 sequences)

SEQ ID NO: 1 - protein, 107 aa, Artificial Sequence [sequence omitted]

SEQ ID NO: 2 - protein, 121 aa, Artificial Sequence [sequence omitted]

SEQ ID NO: 3 - protein, 9 aa, Artificial Sequence
  D2E7 VL CDR3
QRYNRAPYX

SEQ ID NO: 4 - protein, 12 aa, Artificial Sequence
  D2E7 VH CDR3
VSYLSTASSLDX

SEQ ID NO: 5 - protein, 7 aa, Artificial Sequence
  D2E7 VL CDR2
AASTLQS

SEQ ID NO: 6 - protein, 17 aa, Artificial Sequence
  D2E7 VH CDR2
AITWNSGHIDYADSVEG

SEQ ID NO: 7 - protein, 11 aa, Artificial Sequence
  D2E7 VL CDR1
RASQGIRNYLA

SEQ ID NO: 8 - protein, 5 aa, Artificial Sequence
  D2E7 VH CDR1
DYAMH

SEQ ID NO: 9 - protein, 107 aa, Artificial Sequence
  2SD4 VL
DIQMTQSPSSLSASIGDRVTITCRASQGIRNYLAWYQQKPGKAPKLLIYAASTLQSGVPSRFSGSGSGTDFTLTISSLQPEDVATYYCQKYNSAPYAFGQGTKVEIK

SEQ ID NO: 10 - protein, 121 aa, Artificial Sequence
  2SD4 VH
QVQLVESGGGLVQPGRSLRLSCAASGFTFDDYAMHWVRQAPGKGLDWVSAITWNSGHIDYADSVEGRFAVSRDNAKNALYLQMNSLRPEDTAVYYCTKASYLSTSSSLDNWGQGTLVTVSS

SEQ ID NO: 11 - protein, 9 aa, Artificial Sequence
  2SD4 VL CDR3
QKYNSAPYA

SEQ ID NO: 12 - protein, 9 aa, Artificial Sequence
  EP B12 VL CDR3
QKYNRAPYA

SEQ ID NO: 13 - protein, 9 aa, Artificial Sequence
  VL10E4 VL CDR3
QKYQRAPYT

SEQ ID NO: 14 - protein, 9 aa, Artificial Sequence
  VL100A9 VL CDR3
QKYSSAPYT

SEQ ID NO: 15 - protein, 9 aa, Artificial Sequence
  VLL100D2 VL CDR3
QKYNSAPYT

SEQ ID NO: 16 - protein, 9 aa, Artificial Sequence
  VLL0F4 VL CDR3
QKYNRAPYT

SEQ ID NO: 17 - protein, 9 aa, Artificial Sequence
  LOE5 VL CDR3
QKYNSAPYY

SEQ ID NO: 18 - protein, 9 aa, Artificial Sequence
  VLLOG7 VL CDR3
QKYNSAPYN

SEQ ID NO: 19 - protein, 9 aa, Artificial Sequence
  VLLOG9 VL CDR3
QKYTSAPYT

SEQ ID NO: 20 - protein, 9 aa, Artificial Sequence
  VLLOH1 VL CDR3
QKYNRAPYN

SEQ ID NO: 21 - protein, 9 aa, Artificial Sequence
  VLLOH10 VL CDR3
QKYNSAAYS

SEQ ID NO: 22 - protein, 9 aa, Artificial Sequence
  VL1B7 VL CDR3
QQYNSAPDT

SEQ ID NO: 23 - protein, 9 aa, Artificial Sequence
  VL1C1 VL CDR3
QKYNSDPYT

SEQ ID NO: 24 - protein, 9 aa, Artificial Sequence
  VL0.1F4 VL CDR3
QKYISAPYT

SEQ ID NO: 25 - protein, 9 aa, Artificial Sequence
  VL0.1H8 VL CDR3
QKYNRPPYT

SEQ ID NO: 26 - protein, 9 aa, Artificial Sequence
  LOE7.A VL CDR3
QRYNRAPYA

SEQ ID NO: 27 - protein, 12 aa, Artificial Sequence
  2SD4 VH CDR3
ASYLSTSSSLDN

SEQ ID NO: 28 - protein, 12 aa, Artificial Sequence
  VH1B11 VH CDR3
ASYLSTSSSLDK

SEQ ID NO: 29 - protein, 12 aa, Artificial Sequence
  VH1D8 VH CDR3
ASYLSTSSSLDY

SEQ ID NO: 30 - protein, 12 aa, Artificial Sequence
  VH1A11 VH CDR3
ASYLSTSSSLDD

SEQ ID NO: 31 - protein, 12 aa, Artificial Sequence
  VH1B12 VH CDR3
ASYLSTSFSLDY

SEQ ID NO: 32 - protein, 12 aa, Artificial Sequence
  VH1E4 VH CDR3
ASYLSTSSSLHY

SEQ ID NO: 33 - protein, 12 aa, Artificial Sequence
  VH1F6 VH CDR3
ASFLSTSSSLEY

SEQ ID NO: 34 - protein, 12 aa, Artificial Sequence
  3C-H2 VH CDR3
ASYLSTASSLEY

SEQ ID NO: 35 - protein, 12 aa, Artificial Sequence
  VH1-D2.N VH CDR3
VSYLSTASSLDN